(19) United States
(12) Patent Application Publication
(10) Pub. No.: US 2021/0062353 A1
(43) Pub. Date: Mar. 4, 2021

(54) LEVELING AGENT AND COPPER PLATING COMPOSITION COMPRISING THE SAME

(71) Applicant: SOULBRAIN CO., LTD., Seongnam-si (KR)

(72) Inventors: Jong Cheol YUN, Suwon-si (KR); Wan Joong KIM, Ansan-si (KR); Hee Jeong RYU, Suwon-si (KR)

(21) Appl. No.: 17/094,808

(22) Filed: Nov. 10, 2020

Related U.S. Application Data
(62) Division of application No. 16/039,343, filed on Jul. 19, 2018.

(30) Foreign Application Priority Data
Nov. 28, 2017 (KR) 10-2017-0159837

(51) Int. Cl. C25D 3/38; C07D 213/83; C07D 213/59; C07D 213/40

(52) U.S. Cl. CPC C25D 3/38; C07D 213/83; H01L 21/2885; C07D 213/40; C07D 213/59

(57) ABSTRACT

The present invention relates to a A copper plating composition, comprising a metal ion supply source, an electrolyte, and a leveling agent comprising a thioamide compound that comprises at least one of a homocyclic aromatic ring and a heterocyclic aromatic ring. Excellently smooth plated surfaces with minimized defects can be obtained when a feature formed on a substrate are plated by using the copper plating composition.

This application is a divisional application of U.S. patent application Ser. No. 16/039,343 filed on Jul. 19, 2018, which claims priority under 35 U.S.C 119(a) to Korean Patent Application No. 10-2017-0159837 filed on Nov. 28, 2017, the disclosure of which is hereby incorporated by reference in its entirety.

### FIELD OF THE INVENTION

The present invention relates to a leveling agent used for electroplating and a copper plating composition comprising the same.

### BACKGROUND OF THE INVENTION

As interconnections become multilayered upon the manufacture of a semiconductor device, features with high aspect ratios, e.g., vias or trenches, are formed on a substrate. The features are charged by electroplating a composition. At this time, to minimize defects such as voids and seams, additives such as accelerators, suppressors, or leveling agents may be included in the electroplating composition.

During the process of electroplating using the electroplating composition containing accelerator, bumps are formed due to the presence of the accelerator. As the plating process proceeds, an aggregation of bumps is formed as the bumps grow. At such a time, due to the accelerated formation of the bumps on regions with a high aspect ratio and high density, like the features, a bigger aggregation is formed. Such a phenomenon is called overplating. The areas where the overplating has occurred forms steps from the surrounding regions, and the formed steps cause defects in semiconductor devices due to increase of the processing time during chemical mechanical polishing process and impedance of surface smoothness.

Accordingly, a leveling agent is added to increase the smoothness of the surface. Conventionally, the use of polyethyleneimine, polyglycine, polyurea, polyacrylamide, polyaminoamide, polyalkanolamine, and so forth as leveling agents has been disclosed. In addition, the use of copolymers of polyvinylpyridine, polyvinylpyrrolidone, vinylimidazole, vinylpyrrolidone, and so on as leveling agents has also been disclosed (Refer to Patent Literature 1 to 3 below).

However, such leveling agents still have limits to increase surface smoothness. Accordingly, there is a demand for copper electroplating additives, which can excellently interact with accelerators, suppressors, etc., and which can improve the smoothness of a plated surfaces.

Patent Literature 1: WO 2005/066391 A1

Patent Literature 2: US 2003/0168343 A1

Patent Literature 1: US 2006/207886 A1

### SUMMARY OF THE INVENTION

It is an object of the present invention to provide a leveling agent capable of improving the smoothness of a plating surface to solve the aforementioned problems.

It is another object of the present invention to provide a copper plating composition including the leveling agent.

To solve the aforementioned problems, the present invention provides a leveling agent comprising a thioamide compound that comprises at least one of a homocyclic aromatic ring and a heterocyclic aromatic ring.

In addition, the present invention provides a copper plating composition, comprising a metal ion supply source, an electrolyte, and the leveling agent.

### BRIEF DESCRIPTION OF THE DRAWINGS

FIGS. 1A, 1B, 2A, 2B, 3A, and 3B are scanning electron microscope (SEM) images of substrates electroplated by using a copper plating composition in accordance with examples and comparative examples of the present invention wherein FIGS. 1A, 2A, and 3A represent examples 1, 2, and 3, respectively, and FIGS. 1B, 2B, and 3B represent comparative examples 1, 2, and 3, respectively.

### DETAILED DESCRIPTION OF THE PREFERRED EMBODIMENTS

The present invention is described below in detail.

1. Leveling Agent

One aspect of the present invention provides a leveling agent.

The leveling agent controls the smoothness of a plated surface during a plating process. The leveling agent may include a thioamide compound comprising at least one of a homocyclic aromatic ring and a heterocyclic aromatic ring. The homocyclic aromatic ring may include a monocyclic ring with 5 to 6 members or a fused ring with 8 to 14 members whose main ring is composed only of carbon atoms, and the heterocyclic aromatic ring may include a monocyclic ring with 5 to 6 members or a fused ring with 8 to 14 members whose main ring may be composed of nitrogen atoms or sulfur atoms in addition to the carbon atoms.

More specifically, the leveling agent is not limited here, but may be a thioamide compound that comprises at least one of a homocyclic aromatic ring-derived phenyl group and a heterocyclic aromatic ring-derived pyridyl group.

As an example, a phenyl group-containing thioamide compound may be a compound represented as chemical formula 1, shown below.

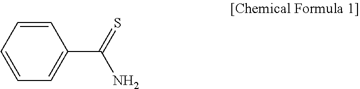

As an example, a pyridyl group-containing thioamide compound may include at least one of the compounds represented as chemical formulas 2 and 3, shown below.

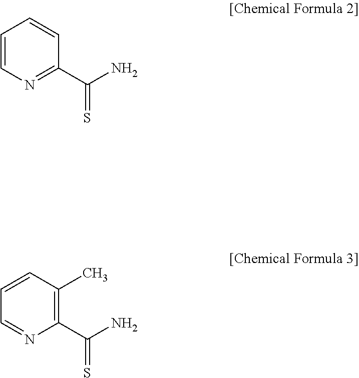

As an example, the phenyl group and the pyridyl group-containing thioamide compound may be a compound represented as chemical formula 4, shown below.

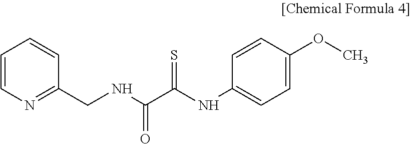 [SMILES: COc1ccc(NC(=S)C(=O)NCc2ccccn2)cc1]

As a thioamide compound which comprises at least one of such phenyl group and pyridyl group, the leveling agent can increase the smoothness of a surface by being adsorbed to the copper surface due to the thioamide functional group.

2. Copper Plating Composition

The other aspect of the present invention provides a copper plating composition.

The copper plating composition includes a metal ion supply source, an electrolyte, and the leveling agent described in Clause 1 above.

The metal ion supply source supplies metal ions during a plating process. Such metal ion supply source may be copper salt, but is not limited thereto. As an example, the copper salt may be copper sulfate, copper chloride, copper acetate, copper citrate, copper nitrate, copper fluoroborate, copper methane sulfonate, copper phenyl sulfonate, copper p-toluene sulfonate, or their mixtures.

The content of the metal ion supply source is not limited here, but in consideration of plating efficiency, it is desirable that it would be from 0.5 to 10% by weight based on the gross weight of the copper plating composition, and it would be more desirable that it makes up 1 to 7% by weight.

The electrolyte gives conductivity to a copper plating composition. It is desirable that such an electrolyte be acidic, and more specifically, it may contain sulfuric acid, acetic acid, fluoroboric acid, methane sulfonic acid, ethane sulfonic acid, propane sulfonic acid, trifluoromethane sulfonic acid, benzene sulfonic acid, p-toluene sulfonic acid, sulfamic acid, hydrochloric acid, hydrobromic acid, perchloric acid, nitric acid, chromic acid, phosphoric acid, or their mixtures.

The content of the electrolyte is not limited here, but in consideration of plating efficiency, it is desirable that it would be from 0.5% to 10% by weight based on the gross weight of the copper plating composition, and it is more desirable that it makes up 1 to 5% by weight.

The leveling agent controls the smoothness of a plated surface during a process of copper plating. A description of the leveling agent is omitted here because it is the same as what has been described in Clause 1, ‘Leveling agent,’ above.

The content of the leveling agent is not limited here, but in consideration of plating efficiency, it is desirable that it would be from 0.00005 to 0.1% by weight based on the gross weight of the copper plating composition, and it is more desirable that it makes up 0.0001 to 0.005% of the content by weight. If the content of the leveling agent is less than 0.00005% by weight, leveling performance may not be good, and if the content of the leveling agent content exceeds 0.1% by weight, smoothness may not be good.

Because the copper plating composition of the present invention includes the leveling agent, if electroplating is carried out using it, excellently smooth plating of the surface can be obtained.

Meanwhile, a representative copper plating composition of the prevent invention may further comprise an accelerator and a suppressor to increase the electroplating efficiency.

The accelerator increases the plating speed of the metal ions during a plating process. Such an accelerator is not limited here, but some specific examples may include N, N-dimethyl-dithiocarbamic acid-(3-sulfopropyl) ester, 3-mercapto-propylsulfonic acid-(3-sulfopropyl) ester, 3-mercapto-propylsulfonic acid sodium salt, bis-sulfopropyl-disulfide, bis-(sodium sulfopropyl)-disulfide, 3-(benzothiazolyl-s-thio) propylsulfonic acid sodium salt, pyridinium propyl sulfobetaine, 1-sodium-3-mercaptopropane-1-sulfonate, N, N-dimethyl-dithiocarbamic acid-(3-sulfoethyl) ester, 3-mercapto-ethylprophyl sulfonic acid-(3-sulfoethyl) ester, 3-mercapto-ethylsulfonic acid sodium salt, bis-sulfoethyl disulfide, 3-(benzothiazolyl-s-thio) ethylsulfonic acid sodium salt, pyridinium ethyl sulfobetaine, 1-sodium-3-mercaptoethane-1-sulfonate, or their mixtures.

The content of the accelerator is not limited here, but in consideration of plating efficiency, it is desirable that it would be from 0.00005 to 0.01% by weight based on the gross weight of the copper plating composition, and it is more desirable that it makes up 0.0001 to 0.005% of the content by weight. If the content of the accelerator is less than 0.00005% by weight, gloss of the plating surface may be reduced, and if the content of the accelerator exceeds 0.01% by weight, the formation of pits could occur, or adhesion may become weak.

The suppressor suppresses the reduction speed of metal ions during the plating process, thereby controlling the speed of plating metal ions. Such a suppressor is not limited here, but specific examples may include Polyethylene Glycol, Polypropylene Glycol, Polyethylene Glycol monoamine, Polypropylene Glycol monoamine, Polyethylene Glycol diamine, Polypropylene Glycol diamine, Polyethylene Glycol monothiol, Polypropylene Glycol monothiol, Polyethylene Glycol dithiol, Polypropylene Glycol dithiol, Polyethylene Glycol monoalkylether, Polypropylene Glycol monoalkylether, Polyethylene Glycol dialkylether, Polypropylene Glycol dialkylether, and at least one selected from copolymer of Ethylene Oxide and Propylene Oxide.

The Content of the suppressor is not limited here, but in consideration of plating efficiency, it is desirable that it would be from 0.001 to 0.1% by weight based on the gross weight of the copper plating composition, and it is more desirable that it makes up 0.005 to 0.05% of the content by weight. If the content of the suppressor is less than 0.001% by weight, uniformity of the plating may not be good, and if the content of the suppressor exceeds 0.1% by weight, the plating speed could be reduced.

In particular, if electroplating is performed on a feature formed on the substrate with a gap size of 5 to 200 nm and an aspect ratio of 2 to 10, the copper plating composition of the present invention could achieve excellent smoothness.

The copper plating composition of the present invention may further comprise a solvent, and it is desirable that the solvent be distilled water (DIW).

In addition, the copper plating composition of the prevent invention may further optionally comprise additives such as a surfactant, an antifoaming agent, or a pH regulator, which are publicly known in the art.

A detailed description of the present invention will be made through examples below. However, the following examples are just examples of the present invention, and the present invention is not limited thereto.

### Example 1

Copper Plating Liquid

A plating liquid was made by stirring and at the same time dissolving the substances listed below with DIW as a solvent. The composition of the plating liquid is as follows:

Copper ion source: Copper sulfate (4 wt %)

Electrolyte: Sulfuric acid (1 wt %)

Chloric ion source: hydrochloric acid (0.005 wt %)

Accelerator: Bis-sulfopropyl-disulfide (0.001 wt %)

Suppressor: PEG (Mw 2000) (0.02 wt %)

Leveling agent: 2-pyridine thioamide (Chemical formula 2) (0.001 wt %)

Solvent: DIW (remaining portion)

Electrolytic Copper Plating

A silicon wafer where trenches with the depth of 370 nm and the diameter of 90 nm are formed was dipped in the copper plating liquid prepared as described above and then plating was conducted by applying a current with a current density of 1.0 ASD. Upon plating, the temperature of the plating liquid was maintained at 25° C. and stirring was performed continuously.

### Example 2

Except that 2-pyridine thioamide (chemical formula 2) (0.0001 wt %) was used as the leveling agent, a copper plating liquid and electrolytic copper plating process were implemented by the same method as in Example 1.

### Example 3

Except that 2-pyridine thioamide (chemical formula 2) (0.0005 wt %) was used as the leveling agent, a copper plating liquid and electrolytic copper plating process were implemented by the same method as in Example 1.

Except that 2-pyridine thioamide (chemical formula 2) (0.0005 wt %) was used as the leveling agent, a copper plating liquid and electrolytic copper plating process were implemented by the same method as in Example 1.

### Comparative Example 1

Except that benzotriazole (0.001 wt %) was used as the leveling agent, a copper plating liquid and electrolytic copper plating process were implemented by the same method as in Example 1.

### Comparative Example 2

Except that benzotriazole (0.0001 wt %) was used as the leveling agent, a copper plating liquid and electrolytic copper plating process were implemented by the same method as in Example 1.

### Comparative Example 3

Except that benzotriazole (0.0005 wt %) was used as the leveling agent, a copper plating liquid and electrolytic copper plating process were implemented by the same method as in Example 1.

### Experimental Example

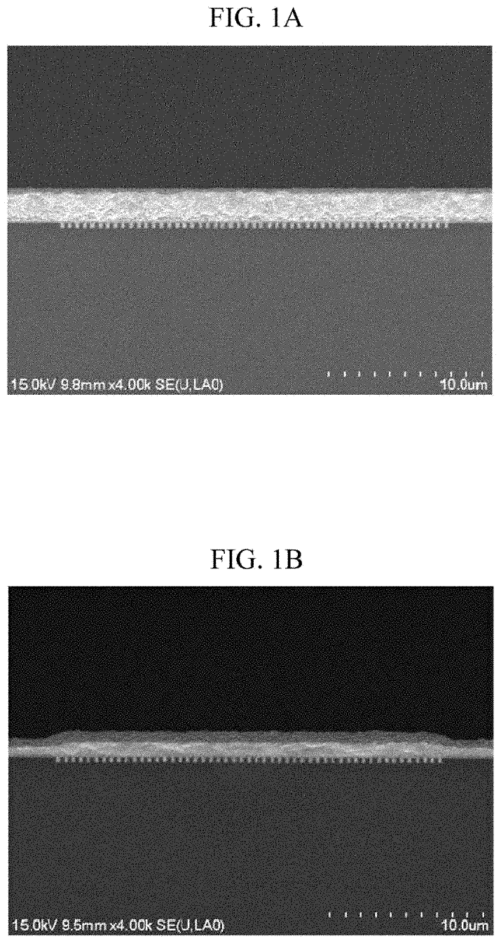

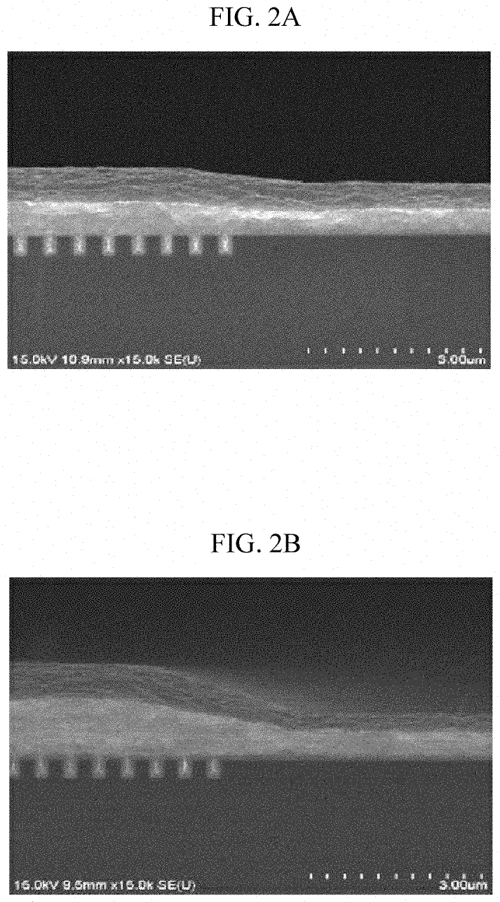

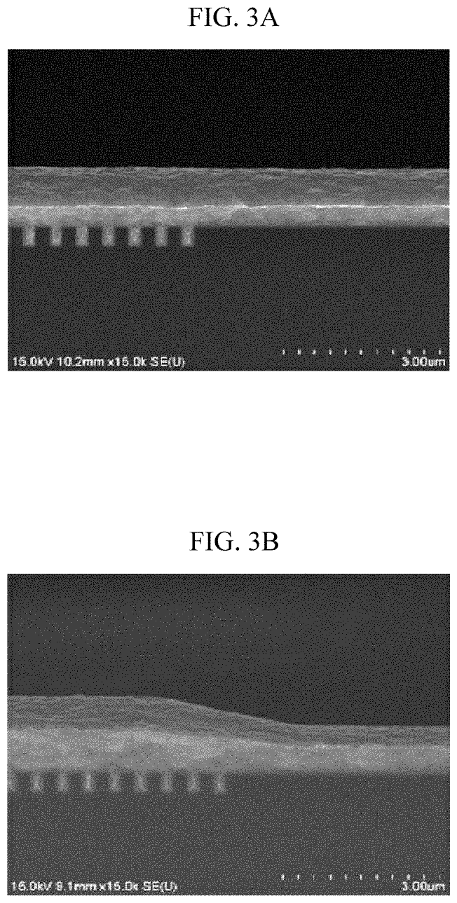

1) After sections of the individual silicon substrates electroplated with copper in accordance with the representative and comparative examples were cut, the state of copper filled (charged) in each of the trenches was observed through a scanning electron microscope (SEM), the results of which are illustrated in FIG. 1 to 3.

2) In plating baths where the examples and comparative examples of electroplating compositions were respectively filled, 90 nm-patterned wafers with patterns of 1:4 aspect ratio were inserted, and electroplating was conducted at 1 ASD for 5 minutes. After electroplating, the leveling of the patterned wafers was checked. Ratios of H1 to H2 were as shown in Table 1 below, where the inside of a hall (pattern) is H1 and the outside thereof is a bulk area (H2). (A ratio close to 1 indicates that the surface is leveled more smoothly.)

TABLE 1

| | | | | Comparative | Comparative | Comparative |
|---|---|---|---|---|---|---|
| | Example 1 | Example 2 | Example 3 | Example 1 | Example 2 | Example 3 |
| Leveling | 0.97 | 1.30 | 1.03 | 1.32 | 2.05 | 1.62 |

Referring to the Table 1 and FIGS. 1 to 3, the copper plating composition of the present invention, which included a leveling agent comprising a pyridyl group-containing thioamide compound, was found to obtain much better smoothness compared to that displayed by the other comparative examples, which did not contain such a compound.

When electroplating is performed using the copper plating composition which includes the leveling agent of the present invention, excellent smoothness of the plating surface can be obtained. Accordingly, the present invention can provide substrates much more uniformly plated with minimized defects, which may improve the reliability of semiconductor devices.

What is claimed is:

1. A copper plating composition, comprising:
a metal ion supply source,
an electrolyte, and
a leveling agent comprising a thioamide compound that comprises at least one of a homocyclic aromatic ring and a heterocyclic aromatic ring.

2. The copper plating composition of claim 1, wherein content of the leveling agent is from 0.00005 to 0.1% by weight based on the gross weight of the copper plating composition.

3. The copper plating composition of claim 1, further comprising an accelerator and a suppressor.

4. The copper plating composition of claim 3, wherein the accelerator comprises N, N-dimethyl-dithiocarbamic acid-(3-sulfopropyl) ester, 3-mercapto-propylsulfonic acid-(3-sulfopropyl) ester, 3-mercapto-propylsulfonic acid sodium salt, bis-sulfopropyl-disulfide, bis-(sodium sulfopropyl)-disulfide, 3-(benzothiazolyl-s-thio) propylsulfonic acid sodium salt, pyridinium propyl sulfobetaine, 1-sodium-3-mercaptopropane-1-sulfonate, N, N-dimethyl-dithiocarbamic acid-(3-sulfoethyl) ester, 3-mercapto-ethylprophyl sulfonic acid-(3-sulfoethyl) ester, 3-mercapto-ethylsulfonic acid sodium salt, bis-sulfoethyl disulfide, 3-(benzothiazolyl-s-thio) ethylsulfonic acid sodium salt, pyridinium ethyl sulfobetaine, 1-sodium-3-mercaptoethane-1-sulfonate, or their mixtures.

5. The copper plating composition of claim 3, wherein the accelerator comprises Poly Ethylene Glycol, Poly Propylene Glycol, Polyethylene Glycol monoamine, Polypropylene Glycol monoamine, Polyethylene Glycol diamine, Polypropylene Glycol diamine, Polyethylene Glycol monothiol, Polypropylene Glycol monothiol, Polyethylene Glycol dithiol, Polypropylene Glycol dithiol, Polyethylene Glycol monoalkylether, Polypropylene Glycol monoalkylether, Polyethylene Glycol dialkylether, Polypropylene Glycol dialkylether, and at least one selected from copolymer of Ethylene Oxide and Propylene Oxide.

6. The copper plating composition of claim 1, wherein the thioamide compound comprises a phenyl group-contained thioamide compound.

7. The copper plating composition of claim 1, wherein the thioamide compound comprises a compound represented as chemical formula 1 as shown below.

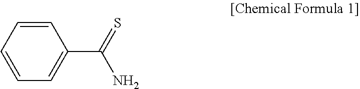

8. The copper plating composition of claim 1, wherein the thioamide compound comprises a pyridyl group-containing thioamide compound.

9. The copper plating composition of claim 1, wherein the thioamide compound comprises at least one of compounds represented as chemical formulas 2 and 3 as shown below.

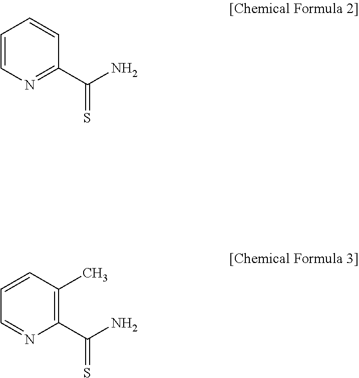

10. The copper plating composition of claim 1, wherein the thioamide compound comprises a compound represented as chemical formula 4 as shown below. 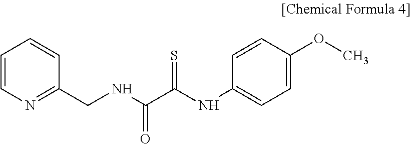 [SMILES: COc1ccc(NC(=S)C(=O)NCc2ccccn2)cc1]
